(19) United States
(12) Patent Application Publication
(10) Pub. No.: US 2017/0035508 A1
(43) Pub. Date: Feb. 9, 2017

(54) COHERENT IMAGING FIBER BASED HAIR REMOVAL DEVICE

(71) Applicant: The Research Foundation for The State University of New York, Albany, NY (US)

(72) Inventor: Harbans S. DHADWAL, Setauket, NY (US)

(73) Assignee: The Research Foundation for The State University of New York

(21) Appl. No.: 15/333,489

(22) Filed: Oct. 25, 2016

Related U.S. Application Data
(63) Continuation-in-part of application No. 12/246,097, filed on Oct. 6, 2008, now Pat. No. 9,474,576.
(60) Provisional application No. 60/977,851, filed on Oct. 5, 2007.

(51) Int. Cl. A61B 18/20; A61B 90/00; A61B 18/22

(52) U.S. Cl. CPC A61B 18/203; A61B 18/22; A61B 90/361; A61B 2018/2205; A61B 2018/2244; A61B 2018/225; A61B 2090/3614

(57) ABSTRACT

Provided are an apparatus and method for hair removal. The apparatus includes a light source, a coherent imaging fiber, a plurality of multimode optical fibers that transmit energy from the light source, and an applicator housing each proximal end of each optical fiber of the plurality of multimode optical fibers, with the coherent imaging fiber transmitting an image of a hair follicle from among a plurality of hair follicles, for viewing on a display.

### PRIORITY

This application is a Continuation In Part application of U.S. application Ser. No. 12/246,097, filed Oct. 6, 2008, and issued as U.S. Pat. No. 9,474,576 on Oct. 25, 2016, and claims priority to U.S. Provisional Application No. 60/977,851, filed Oct. 5, 2007, the content of each of which is incorporated herein by reference.

### BACKGROUND OF THE INVENTION

1. Field of the Invention

The present invention relates generally to selective and extended photothermolysis for cosmetic, health and dermatology conditions, and more particularly, to a portable device for photo-inducing damage to cellular structures for hair removal and method for operation of same.

2. Brief Description of the Background Art

Electromagnetic energy, particularly in the optical band of 400 nm to 1200 nm, has been used for treatment of many skin related diseases as well as for cosmetic procedures, e.g., hair removal, spider veins, tattoos, port wine stains, skin rejuvenation and photodynamic therapy. Laser and light-based removal of hair, both in men and women, is widely accepted as a successful approach. In today's market place, manufacturers have focused on four laser-based systems: alexandrite (755 nm); neodymium-doped yttrium aluminum garnet (1064 nm); laser diodes (810 nm); and a broad band intense pulsed light (IPL) source. Generally, these systems provide reduction in the growth cycle of hair. Multiple treatments have been found to improve upon longevity of the hair free period. An endpoint for an acceptable treatment requires destruction of pleuripotential follicular stem cells and not merely evaporation of the hair shaft.

Recent data suggests that stem cells are found in upper bulb and bulge regions of the hair follicle. Indeed there may be other areas not yet identified. Laser hair removal (LHR) procedures target these regions of the stem cells, as they are responsible for hair growth. Several techniques have been developed for destruction of stem cells.

Laser ablation, not typically used for photoepilation, uses high energy short pulses to raise the temperature of the stem cell above that required for evaporation, however, the target and the absorber must be collocated. Selective photothermolysis exploits dissimilar absorption coefficients of the photo absorbers and surrounding tissue. However, use of selective photothermolysis for destroying the stem cells responsible for hair growth is compounded because the photo-absorbing chromophore, melanin, is found both in the follicular stem cells and the epidermis. Melanin has a broad absorption spectrum and is responsible for pigmentation of the hair shaft and skin. Selective photothermolysis techniques are effective if a concentration of melanin is higher, by a factor of five, in the target area. These techniques work particularly well for dark hair on light skin. However, unavoidable absorption of photons in the epidermis leads to heat, which needs to be removed to avoid damage to the epidermis. Consequently, hand-pieces that chill the epidermis during treatment have been developed.

Destruction of cells through thermal denaturing requires that a target temperature exceed 70° C. within the thermal relaxation time (TRT) of the tissue. For the hair shaft, the TRT is in the range of 35 to 50 ms. Pulse widths exceeding the TRT permit diffusion of heat into surrounding tissue preventing the denaturing temperature from being reached due to heat leakage. Typically, LHR devices target about a 1 cm² area of the skin, which is bombarded with photons. Some photons are absorbed in the epidermis, while the remaining migrate, via scattering, through the dermis and reach the melanin rich hair shaft and bulb region, where absorption leads to elevation of tissue temperature causing cell destruction. The photons scattered in the backward direction return back to the epidermis resulting in fluence levels exceeding the incident fluence.

Based on photon transport theory and clinical data, an optimum set of parameters can be established for a particular device. Unfortunately, these parameters are patient dependent and use of LHR devices remains an art.

A typical laser diode system will have a variable fluence between 20 to 60 J·cm⁻², a pulse width in the range of 5 to 500 ms, and a treatment spot size of ˜1 cm². The peak power of the source, which determines the size of the LHR system, is proportional to the product of fluence and spot area and inversely proportional to the pulse width. For example, a 100 μs pulse with a spot area of 1 cm² requires a peak pulse power of 20 kW for a fluence of 20 J·cm². Consequently, this leads to bulky and expensive machines, which need full medical facilities for operation. While the large diameter reduces treatment time and increases penetration depth into the dermis, it lacks the capability to selectively remove hair from a given area, i.e., to reduce hair density.

Another approach for permanent hair removal is based on extended selective photothermolysis (ESP). The target to be denatured can be separated from a photo-absorber, known as a heat source. A closer study of the underlying thermal diffusive processes has led to use of longer pulses to produce a hot spot in the melanin rich hair shaft. The longer laser pulse produces a hot spot, which begins to heat the surrounding tissue, including the hair bulb and bulge. Pulse width is determined by the TRT and the thermal damage time (TDT). Recent studies have indicated, particularly for techniques using the hair shaft for heat transmission, that a longer pulse width up to 1.5 seconds may be acceptable, which substantially decreases the peak power requirement. Several LHR systems with peak power up to 200 W using laser diode arrays are now on the market.

Other procedures for efficiently using the available photons in LHR devices include a pretreatment that applies highly reflective and thermally conductive applications to the skin prior to laser treatment. Ultrasonic massaging increases penetration of a dye into the epidermis. Pre-treatments can be used with any of the light-based techniques to enhance efficacy of hair removal, but adds extra time and cost to the treatment.

U.S. Pat. No. 7,118,563 to Weckwerth discloses a rechargeable device suitable for providing therapeutic energy. However, the minimum spot size of 0.25 cm² is too large for targeting single hair follicles and causes a reduction in the peak power requirement. The system disclosed by Weckwerth also lacks any imaging device for identifying a treatment area.

U.S. Pat. No. 7,220,254 to Altshchuler teaches that existing technology can be packaged into a self-contained hand-held device for delivery of therapeutic energy to a skin treatment area and can be visualized by an image capturing system integrated into the hand-held device. The device combines discrete optical and electronic components to illuminate an area of the skin to facilitate imaging by a charge coupled device/complementary metal oxide semiconductor (CCD/CMOS) device. Imaging and treatment optical paths are separated by a beam splitter. A more compact and user-friendly hand-held device, with few components, would be more desirable, particularly for the home market.

U.S. Pub. No. 2007/0198004 to Altshchuler et al. addresses some of the above problems in disclosing a tethered hand-piece which may be more appropriate for the home market. However, conventional photo cosmetic devices do not include imaging capability and use lower power EMR sources having prolonged exposure times. For hair removal, such devices recommend power levels in the range of 20-500 W, which is not attainable by a single laser diode.

### SUMMARY OF THE INVENTION

The present disclosure has been made to address at least the above problems and disadvantages, and to provide at least the advantages described below. Accordingly, an aspect of the present disclosure provides an apparatus for hair removal that includes a light source, a coherent imaging fiber, a plurality of multimode optical fibers configured to transmit energy from the light source, and an applicator configured to house each proximal end of each optical fiber of the plurality of multimode optical fibers, with the coherent imaging fiber transmitting an image of a hair follicle from among a plurality of hair follicles, for viewing on a display.

Another aspect of the present disclosure provides a hair removal method that includes positioning an applicator configured to house proximal ends of each optical fiber of a plurality of multimode optical fibers and a proximal end of a coherent imaging fiber above a plurality of hair follicles; viewing, via the coherent imaging fiber, an image of a hair follicle to be removed from among a plurality of hair follicles; and transmitting, from a light source via the plurality of multimode optical fibers, energy to the hair follicle to be removed from among the plurality of hair follicles.

### BRIEF DESCRIPTION OF THE DRAWINGS

The above and other objects, features and advantages of certain embodiments of the present invention will be more apparent from the following detailed description taken in conjunction with the accompanying drawings, in which:

FIG. 1 is a block diagram of a portable laser hair removal system, according to the present invention;

FIG. 2 is a schematic of a fiber optic hand piece applicator of the portable laser hair removal system of FIG. 1, according to an embodiment of the present invention;

FIGS. 3A-3B are cross-sectional views of an optical switch of the portable laser hair removal system of FIG. 1, according to the present invention;

FIG. 4 is a cross-sectional view of a hair follicle with direct illumination of a hair shaft, according to the present invention;

FIG. 5 is a cross-sectional view of a hair follicle with direct illumination of an inner root shaft using a donut beam, according to the present invention;

FIG. 6 is a timing diagram for dual pulse treatment, according to the present invention;

FIG. 7 is a schematic diagram of a dual laser diode illumination scheme, according to the present invention;

FIGS. 8A-8D are schematics for producing a scanning spot on a target, according to the present invention; and

FIG. 9 is a schematic of the fiber optic hand piece applicator, according to the present invention.

### DETAILED DESCRIPTION OF THE PREFERRED EMBODIMENTS

The following detailed description of preferred embodiments of the invention will be made in reference to the accompanying drawings. In describing the invention, explanation about related functions or constructions known in the art are omitted for the sake of clearness in understanding the concept of the invention, to avoid obscuring the invention with unnecessary detail.

Embodiments of the present invention provide a portable and inexpensive apparatus for locating and capturing an image of a small treatment area, typically, about 250 microns in diameter. Further, the apparatus, typically less than 15 mm in diameter, delivers optical energy from a remote source to a target area, preferably smaller than a size of the image. The small size the apparatus is particularly useful for treating areas that require a reduction of hair density and not indiscriminate hair removal. In a preferred embodiment, the apparatus includes a hand piece mounted on a robotic arm for automated laser hair removal.

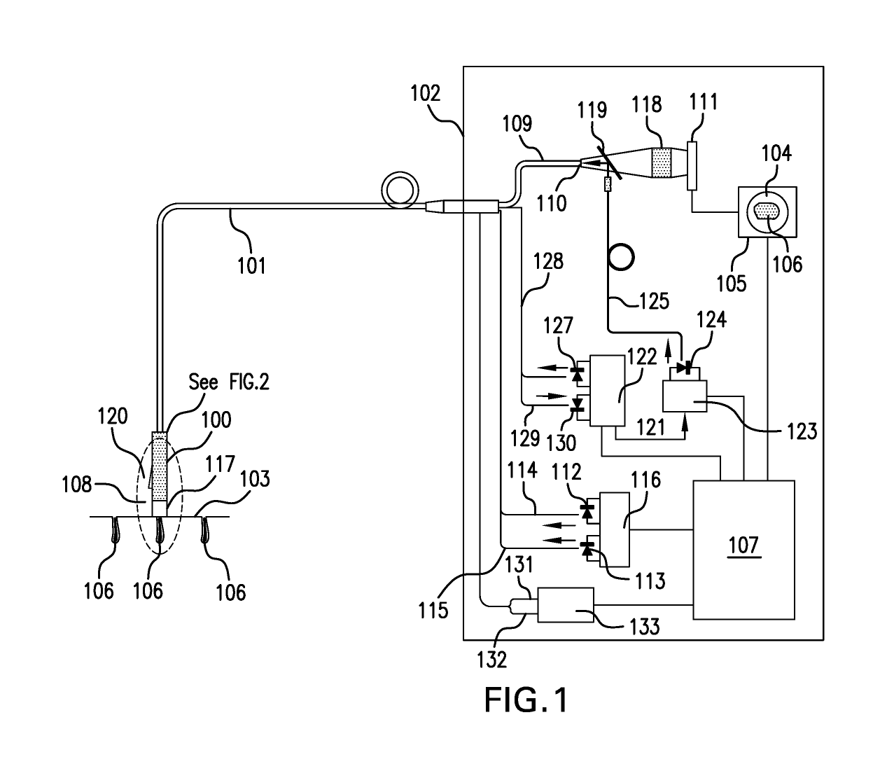

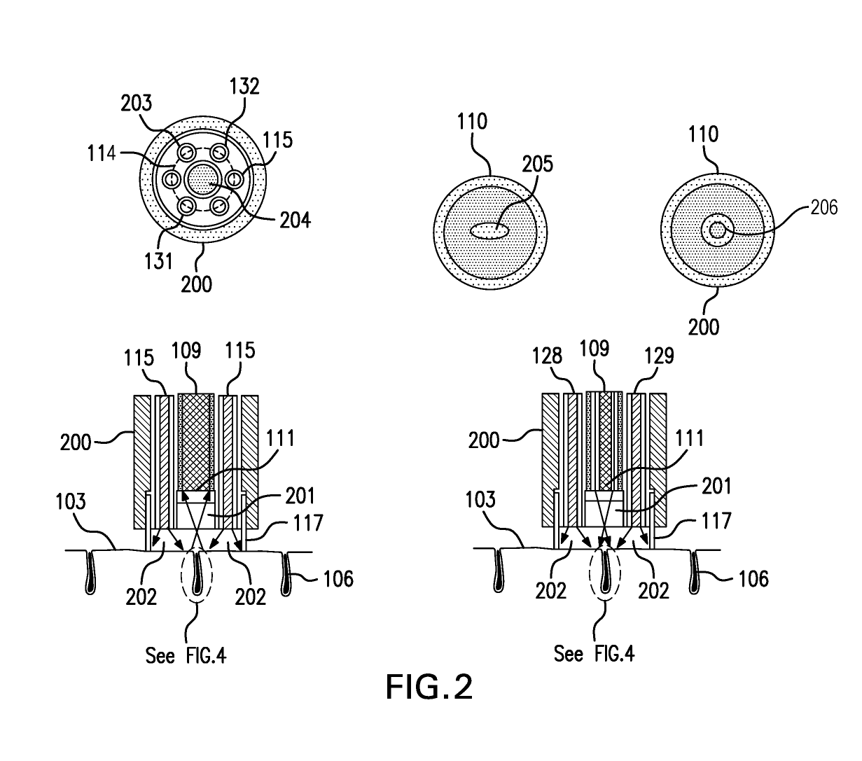

Additionally, the small treatment laser spot can be scanned across the target area to synthesize a larger treatment area. Referring to FIG. 1, a miniature hand piece applicator (HPA) 100 is connected to an electronic console 102 by a flexible umbilical cord 101. The HPA 100 can be manually operated or operated as part of a robotic arm for automated treatment. In a manual mode, the HPA 100 is moved along the surface of a treatment area 103, while viewing a color image 104 on a display 105, until a particular target 106, for example a hair follicle, is located. At this point the color image 104 can be captured and stored in an embedded processor system 107. Referring to FIG. 2, the image capture system includes two optical imaging stages: a first stage using a micro lens 201 to form a primary image of the treatment area 103 at the distal end 109 of the coherent imaging fiber (CIF) 109, which transports a primary image to a proximal end 110 of CIF 109. An aspheric lens forms a magnified image on a CCD sensor 111. Magnification of the primary image is adjustable by axial adjustment of microlens 118. A dichroic beam splitter 119 separates the visible light image from the high energy laser treatment pulse.

Illumination of the treatment surface is achieved by coupling an output from white light emitting diodes 112, 113 to the proximal end of a plurality multimode optical fibers 115, 116, which transport the light to the HPA 100. Intensity of illumination is controlled through control module 122.

An optical system responsible for delivering a high energy therapeutic laser pulse (TLP) to the target uses the same CIF 109 to capture the image of the target. In the manual mode, transmission of the TLP is initiated by a user command, which is generated by an ON/OFF optical switch 120 mounted in the HPA 100. Upon receiving an ON signal from the LED driver assembly, the embedded processor system 107 sends out a programmed series of pulses to a laser diode (LD) driver 123, to power a high power laser diode 124, which is pigtailed to multimode optical fibers 125, the output from the distal end is imaged to the proximal end 110 of the CIF 109 via a source imaging aspheric lens and the dichroic mirror beam splitter 119. The electro-mechanical shutter prevents accidental leakage of the high laser energy, and its operation is synchronized with the ON/OFF pulse. A proximal image of the TLP is transported to the distal end 108 of the CIF 109 in the HPA 100. In this manner, the TLP is delivered precisely to the target 106 with negligible energy leakage beyond the treatment area 103. Pulse parameters are adjustable through the embedded processor system 107.

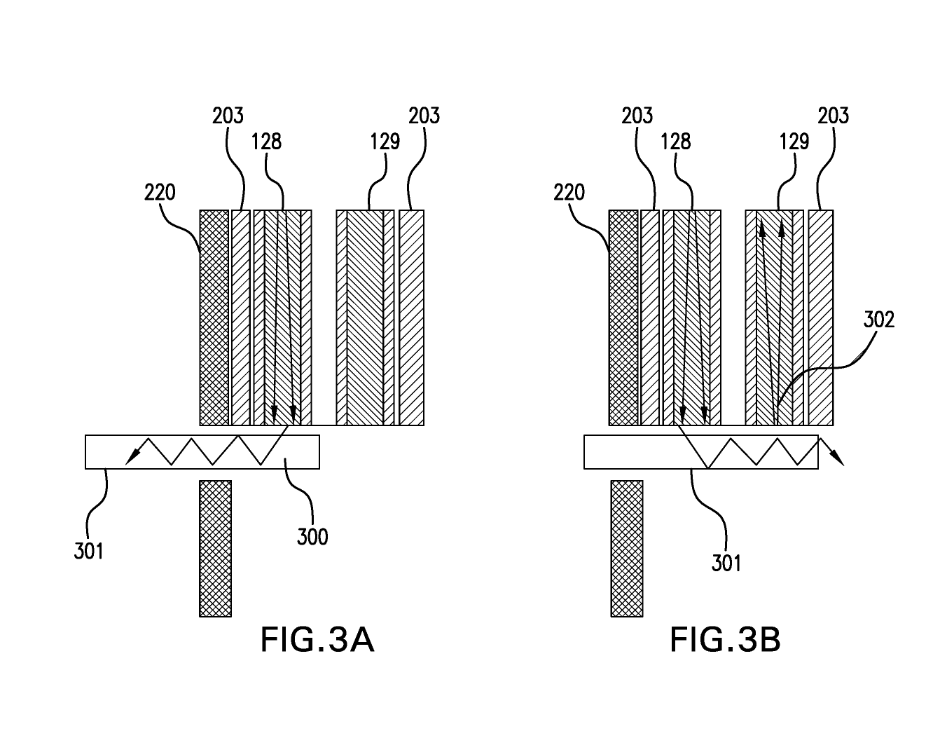

The optical switch 120, also discussed in FIGS. 3A-3B, modulates an optical signal to define ON/OFF states. A modulated signal from the control module 122 drives light emitting diode (LED) 122, pigtailed to optical fiber 125. During the ON state, the modulated optical signal from the HPA 100 is returned to the control module 122 via multimode optical fiber 129 which is pigtailed to a photodetector, e.g., a pin photodiode. The received optical signal 121 is detected and sent to the embedded processor system 107, which uses the ON state to generate the TLP with a preset width and amplitude, and the OFF state is used for shutting down the high power laser diode 124. The ON/OFF signal can be used to provide authentication codes to prevent accidental or unauthorized use of the HPA. The HPA 100 also provides chilled air directed at the treatment area 103. The chilled air is delivered through two stainless steel micro-tubes 131 and 132 from a chilled air source controller 133.

FIG. 2 illustrates a cross-section of the HPA 100 constructed from a cylindrical stainless steel housing 200. The CIF 109 is located in a central region of the HPA 100, surrounded by a ring containing tubing and optical fibers, as discussed below. The CIF 109 typically has a diameter of 700 microns and 50,000 individual pixel elements each having a diameter of 4.5 microns. The distal end 108 of the CIF 109 is positioned in a front conjugate plane of micro lens assembly 201.

As illustrated in FIG. 2, the micro lens assembly 201 forms a de-magnified primary image of the target 106 on the distal end 108 of the CIF 109. The primary image is transported to the proximal end 110. The primary image size can be adjusted by changing the height of the baffle 117. A plurality of multimode optical fibers 114 and 115 provide white light illumination 202, to enhance quality of a captured image. The stainless steel micro-tubes 131 and 132 are used for transporting chilled air to the treatment area 103. Stainless steel conduit 203 is used for holding multimode optical fibers 128 and 129 used in the optical switch 120, while spare stainless steel channel 204 may be used for mounting other sensors, for example, a thermistor for monitoring the target temperature.

During treatment, the CIF 109 delivers the optical energy to the target 106. As illustrated in FIG. 2, arbitrary spatial distributions 205 and 206 are defined by exciting the appropriate pixels at the proximal end 110 of the CIF 109. The micro lens assembly 201 produces the desired spatial image on the target 106. The optical energy is delivered to the treatment area 103 for a programmed precise time and the laser diode 124 is disabled until the optical switch 120 is enabled.

FIGS. 3A-3B provide cross-sectional views of the optical switch 120 in the HPA 100. At least two multimode optical fibers 128 and 129 are mounted in the stainless micro-tubing 203. A modulated optical signal emanates from a distal end of fiber 128. In the OFF state, as illustrated in FIG. 3A, an optical signal 300 enters a slab waveguide 301 and is lost. In the ON state, as illustrated by FIG. 3B, the optical signal 300 enters the slab waveguide 301, and the optical signal leaves the slab waveguide 301, entering a multimode optical fiber 129.

The optical signal is detected by the photodetector 130 (FIG. 1). The optical switch 120 defaults to the OFF state until moved to the ON state by the user. Activation of the optical switch 120 produces a TLP of preset width and repetition rate, thereby providing improved safety.

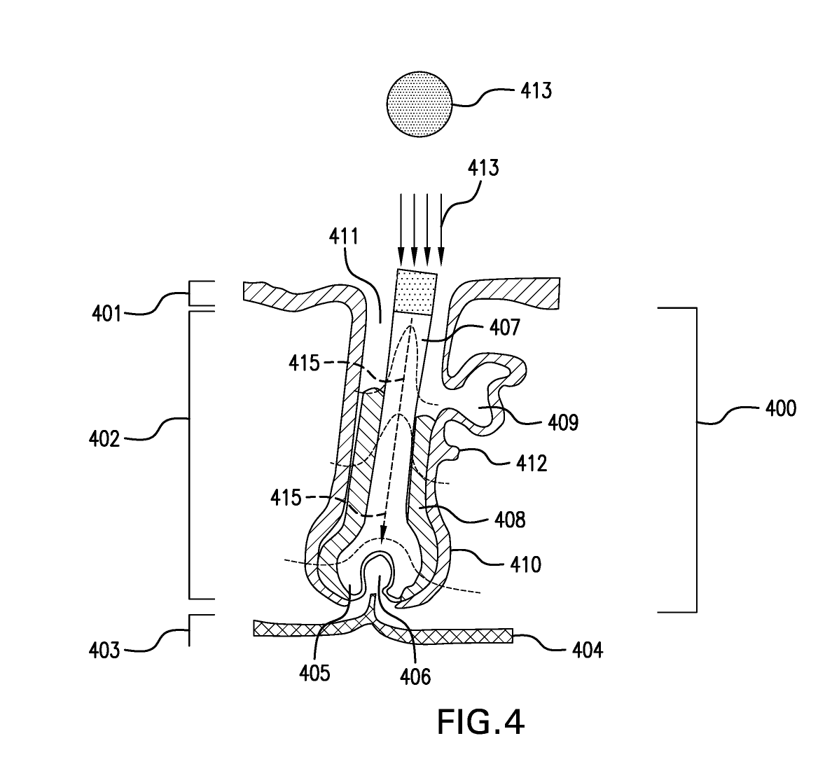

FIG. 4 shows a cross-section of a hair follicle 400, which resides in the following three layers of the skin, i.e., the epidermis 401, the dermis 402, and the hypodermis 403. During the anagen phase of the hair growth cycle, capillaries 404 provide nutrients to the bulb region 405, which encompasses the dermal papilla 406. During this phase of the hair growth cycle, the bulb region 405 is located 2 to 4 mm below the epidermis 401. The hair shaft 407 and the inner root sheath (IRS) 408 grow together from the bulb region 405 upward toward the sebaceous gland 409. Each of the various follicular compartments arises from the germinative cell pool at the base of the bulb region 405. An inner most layer of the outer root sheath (ORS) 410 provides a slippage plane. The ORS 410 remains behind and is continuous with the epidermis 401. The IRS 408 disintegrates just below the sebaceous gland 409 and the sheath-free hair shaft 407 exits the pilary canal 411. The bulge region 412, the putative site of follicular stem cells and the bulb region 405 contain melanocytes, which give the hair shaft 407 its color. The bulge region 412 and the bulb region 405 are the primary targets for photothermolysis as they exhibit a broad absorption spectrum in the visible and near infrared regions. Melanocytes are composed of eumalanin, which is brownish-black, and phuemelanin, which is reddish. Photons from the TLP are delivered to the bulge region 412 and the bulb region 405 in order to cause cell destruction. Selective photothermolysis methods of photoepilation bombard a large area of the epidermis 401 in order to increase the probability of reaching target areas. Photons in the TLP are lost due to reflection at the epidermis 401, absorption in the epidermis 401, and scattering in the dermis 402. The probability of photons reaching intended targets is extremely low, requiring high surface fluence values and large treatment area sizes. In addition, deeper targets, such as the hair follicle 400, are only reachable at longer wavelength (750-1000 nm). However, the absorption of melanin drops of at longer wavelengths, requiring even higher fluences.

Decreasing a requirement for peak power through a reduced spot size of the TLP pulse is not a viable solution as the photons migrate out of the target zone very rapidly. Moving to a smaller spot size demands new delivery methods for reaching the intended targets. Three optical delivery techniques are provided which target individual hair follicles, typically with a spot size smaller than 10⁻⁴ cm². One of these is ESP, which uses heat diffusion to reach the intended targets by creating hot spots in easily accessible parts of the hair follicle, mainly the hair shaft 407. However, delivering the TLP directly to the hair shaft 407, which has a nominal diameter of 80 μm, requires precise spatial location. Imaging and sensor techniques have been proposed for achieving this goal, but all of the proposed solutions include scanning functionality in the hand piece, something that should be avoided if the device is to be utilized in non-medical facilities.

As illustrated in FIG. 4, the hair shaft 407 is illuminated with a Gaussian laser spot 413, with a diameter slightly smaller than a diameter of the hair shaft 407, typically about of 80 μm and the pilary canal 411 has an opening with a nominal diameter of 200 μm. The hair shaft 407 is a highly absorbing medium and has no useful optical guiding properties. In “Characterization of human scalp hairs by optical low-coherence reflectometry,” Opt. Let., 20, 6, 524-526 (1995) by Wang et at., optical low-coherence reflectometry measurements of longitudinal scans of dark and light hair are provided. Wang et al. reported that a refractive index of the hair shaft increased from 1.57 for blond hair to 1.59 for black hair. From the data of Wang et al., an attenuation coefficient for black and blond hair was estimated to be 34.5 mm⁻¹ and 3.2 mm⁻¹, respectively. From these measurements, made at 850 nm, effective penetration depths of 29 μm and 310 μm for black and blond hair, respectively, was determined. These measurements indicate that the hair shaft is not an optical fiber waveguide. Thus, photons incident on the hair shaft are absorbed within this short layer, causing a localized hot spot. By using lower fluences and longer pulse widths (500 ms), the dermal papilla 406 and bulb region 405 can be heated to denaturing temperatures by allowing the heat to diffuse down the hair shaft 407, along longitudinal axis 415.

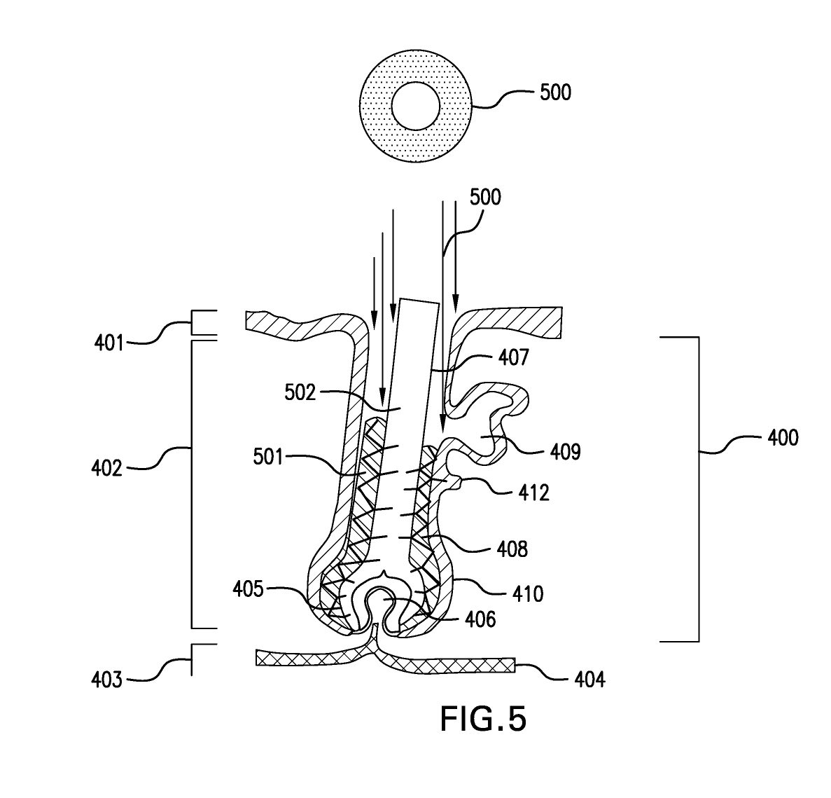

FIG. 5 shows a second illumination strategy, which deposits photons in melanin rich sites of the hair shaft 407 by using an optical guiding channel created by a concentric structure of the hair shaft 407, the IRS 408, and the ORS 410. Specifically, the hair shaft 407, the IRS 408, and the ORS 410 form a three layer waveguide. Entrance to the three layer waveguide is through the pilary canal 411. The IRS 408, which is sandwiched between the ORS 410 and the hair shaft 407, below the sebaceous gland 409, has a refractive index that is larger than that of the ORS 410 but lower than that of the hair shaft 407. The three layers form a leaky waveguide, with the photons being absorbed on a hair shaft surface 502 and reflected from an ORS surface 501. A donut shaped TLP 500 is matched to a size of the pilary canal 411, which has inner diameter bounded by the hair shaft 407 and a nominal ring thickness 30-40 μm. Photons enter the pilary canal 411, which may contain an oil substance excreted by the sebaceous gland 409, enter the IRS 408 below the sebaceous gland 409, and are guided through the leaky modes to the melanin rich bulb region 405 containing the stem cells to be destroyed. As these photons travel in the IRS 408 some are likely to be absorbed by the melanocytes in the bulge region 412. The fluence levels may be lower as none of the incident photons are absorbed by the epidermis 401 or the dermis 402. Consequently the epidermis 401 should experience minimum heat stress. In this configuration pulse widths should correspond to the TRT of the bulb region 405.

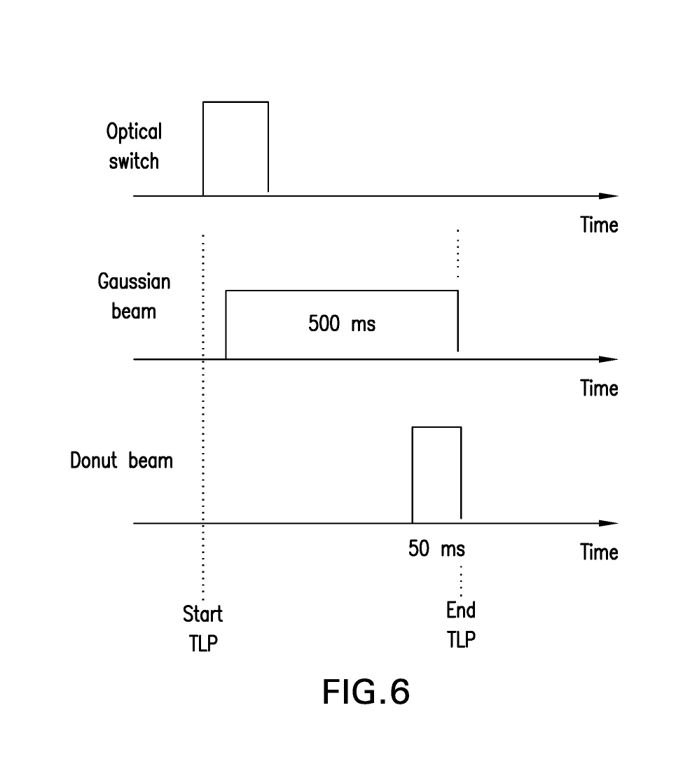

A third illumination strategy can be a combination of both those described above. A short pulse width donut beam can be superimposed on a long pulse width Gaussian beam toward the end of the short pulse width donut beam's duration, as indicated by the timing diagram in FIG. 6. This strategy allows the hair shaft's 407 temperature to be elevated by the extended TLP directed at the hair shaft 407, followed by the donut shaped pulse just prior to the termination of the Gaussian pulse. The Gaussian pulse may have a pulsed width in the range of 100 to 500 ms, while the donut shaped pulse width is between 5 and 50 ms.

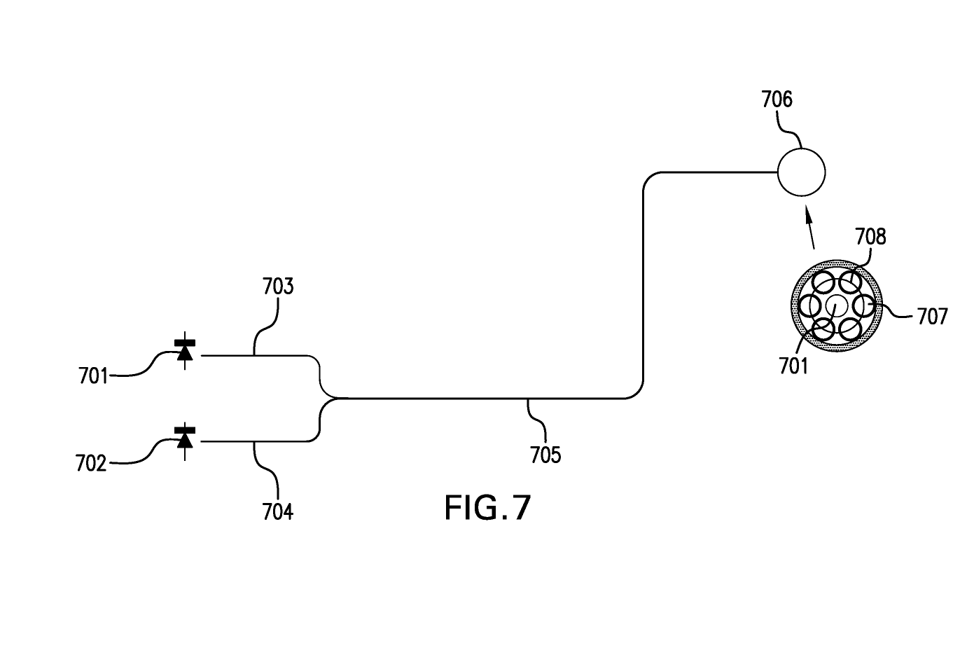

Referring to FIG. 7, a first laser diode (LD1) 701 is pigtailed to a single multimode fiber 703 which forms a central part of the distal end 706 of a fiber optic assembly 705. A second laser diode (LD2) is pigtailed to plurality of multimode fibers 704 which are arranged in an annular pattern 707 forming a circle 708 surrounding the single multimode fiber 705. In this way, LDs 701 and 702 may either have identical or dissimilar spectral and power properties. In principle, the two laser diodes 701 and 702 may have different wavelength and deliver different fluence levels, which could be matched for the hair color.

The distal end 706 can be integrated with an source aspheric imaging lens assembly.

There may be instances when a larger spot is required. As discussed above, with reference to FIGS. 2A and 2B, it is possible to produce any arbitrary illumination shape. For example, an elliptical spot which increases the spot dimension along one axis, while continuing to maintain a small usable area. However, there may be instances when this approach is also not adequate. In such situations a large treatment area can be synthesized by scanning the small spot over the target area. U.S. Pat. No. 7,101,365 to Sharon describes a manual means to pivot an entire hand piece to obtain a limited scan. While Altshchuler and U.S. Pat. No. 5,860,967 to Zavisian include a 2-D scanning mechanism in the hand piece, the present invention achieves desired scanning of the target 106 by scanning an image of the source at the proximal end 110 of the CIF 109.

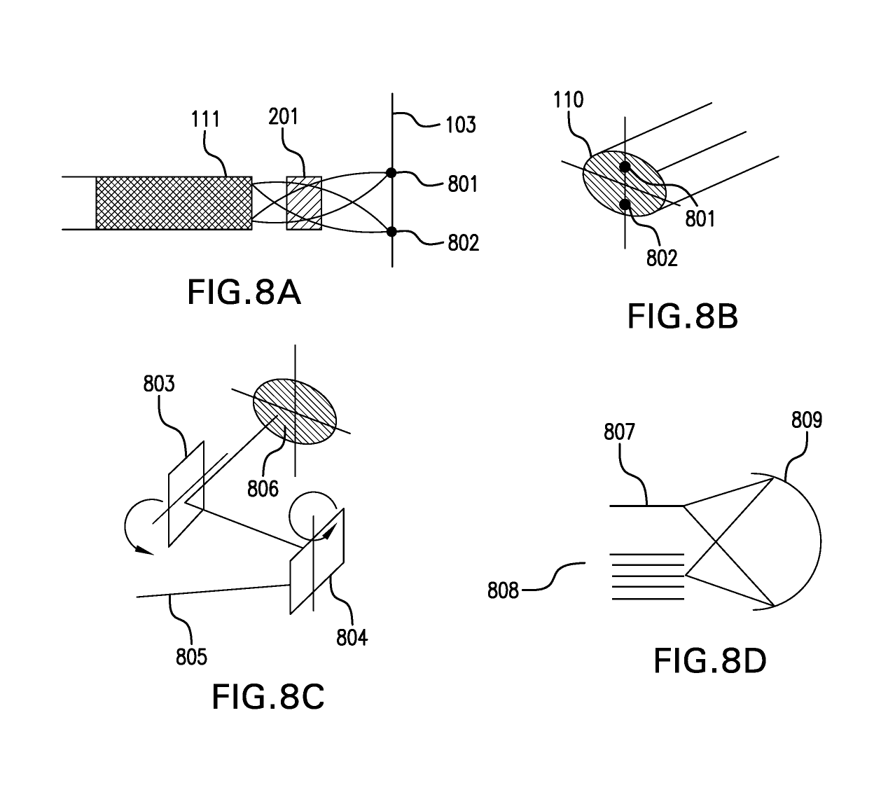

In FIG. 8A, dots 801, 802 show arbitrary positions of the TLP on the treatment area. FIG. 8B shows positions of the corresponding dots 801, 802 on the proximal end 110 of the CIF. A target scan path corresponds to a scanned source image on the proximal end 110 of the CIF.

The scanned image can be generated in a number of ways. FIG. 8C illustrates a two-dimensional mechanical scanner using mirrors 803 and 804. A laser source beam 805 bounces off the mirrors 803 and 804 to define a scan path 806.

FIG. 8D illustrates an alternative method to obtain the source scan, using a 1×N fiber optic switch. In other words, output of an input fiber 807 can be directed to any one of output fibers 808 by means of, for example, a rotating concave mirror 809. Other types of switches may be used. The output fibers 808 form the distal end 706 of the fiber optic bundle 705 described in FIG. 7. A difference between the Sharon and Altshchuler schemes is that all scanning components are in the electronic console 102, none of the scanning components are in the HPA 100. This ensures a compact and safe hand piece suitable for non-medical facilities.

There are certain situations when indiscriminate hair removal using a large diameter spot is not desirable. As an example, for cosmetic purposes, patients may require an alteration of the hair density in certain parts of the human anatomy rather than total hair removal. For such applications a LHR system must be capable of targeting individual hair follicles. The HPA 100 described above can be used on a robotic platform to remove hair from any random location. One such embodiment includes a three-dimensional system that creates a digital map of a surface to be treated. Appropriate software algorithms that analyze hair distribution and hair angle determine optimum location information of hair follicles to receive laser treatment. The location information drives the robotic arm to automatically complete the treatment. Safety features, built around limit switches, ensure that the high energy spot remains within the treatment area.

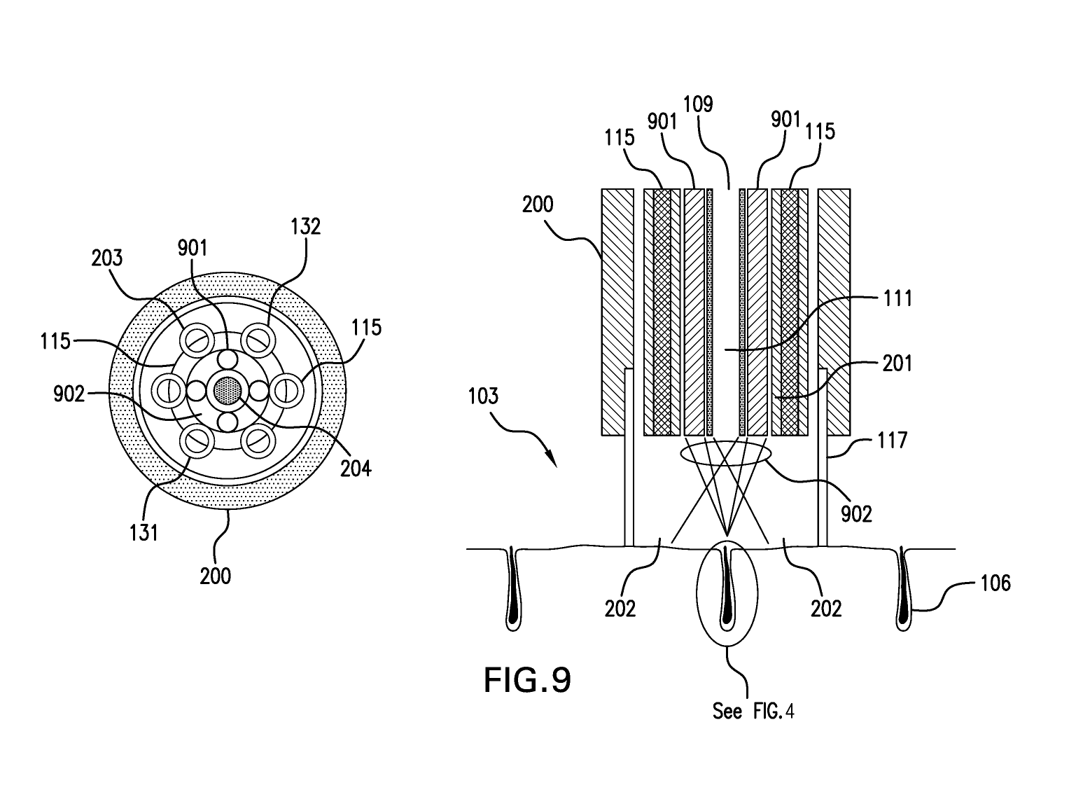

Another preferred embodiment of the HPA 100 is illustrated by FIG. 9. The CIF 109 is surrounded by a plurality of multimode fibers 901 which are used for delivering high energy optical pulses to the hair follicle. Output of the multimode fibers 901 is combined into a single spot at an entrance to the target 106. A radially bi-focal lens 902 provides disparate magnifications for the CIF 109 and the multimode fibers 901. The multimode fibers 901 can be used to increase the target fluence by using a plurality of optical sources at the same emission wavelength, or alternatively, sources with output at different wavelengths could be combined to enhance efficacy of the treatment.

An example of a fluence calculation in a preferred embodiment is as follows. An expected fluence Fₜ [J·cm⁻²] at the target 106 of area Aₜ [cm²] to the power P_{f} emanating from a pigtailed laser diode assembly is given by Equation (1):

$${F}_{t} = η \frac{{P}_{f} {σ}_{T}}{{A}_{T}} \qquad ( 1 )$$

where η represents all the transmission losses from the output of the fiber assembly to the laser spot illuminating the treatment area 106 and σ_{T} is the pulse width or duration of the optical energy pulse which can be easily controlled between 100 μs to 1 s. Using a conservative estimate of η=0.85, P_{f}=200 mW, Aₜ=10⁻⁴ cm², which is a typical diameter of the hair shaft, and σ_{T}=50 ms, Fₜ=85 J·cm² is obtained. Thus, the fluence can be controlled through a combination of parameters, P_{f}, σ_{T}, and A_{T}.

A non-contact temperature sensor detects treatment surface temperature by capturing a portion of the radiated electromagnetic spectrum in the near to mid Infrared region. For example, a Melexis Infrared thermometer, e.g., MLX90615, pigtailed from an imaging fiber, converts emissions in the spectral band of 5.5 μm to 14 μm into a digital code corresponding to temperature in the range of −20° C. to 85° C. The non-contact temperature sensor operates via an optical fiber optimized for transmission in a mid infrared range of wavelengths to guide radiated emission from the treatment surface directly to the sensor. Output of the non-contact temperature sensor is utilized to generate an abort signal to shut-down the high power laser diode 124 when the measured surface temperature exceeds a prescribed temperature threshold. The abort signal disconnects the high power laser diode 124 from driver 123, resulting in the immediate cessation of the treatment. This sensory feedback arrangement, based on continuous temperature monitoring of the treatment surface, prevents burning in the treatment area, and ensures safe operation.

The non-contact temperature sensor can output directly into the applicator hand piece 100 and send the abort signal through the flexible umbilical cord 101 to the high power laser diode 124. However, direct output to the applicator hand piece 100 is not preferred since low current electrical signals are transmitted through the flexible umbilical cord 101, and such signals will be subject to added noise.

While the invention has been shown and described with reference to certain embodiments of the present invention thereof, it will be understood by those skilled in the art that various changes in from and details may be made therein without departing from the spirit and scope of the present invention as defined by the appended claims and equivalent thereof.

What is claimed is:

1. An apparatus for hair removal, the apparatus comprising:
a light source;
a coherent imaging fiber;
a plurality of multimode optical fibers configured to transmit energy from the light source; and
an applicator configured to house each proximal end of each optical fiber of the plurality of multimode optical fibers,
wherein the coherent imaging fiber is configured to transmit an image of a hair follicle from among a plurality of hair follicles, for viewing on a display.

2. The apparatus of claim 1, wherein the light source outputs electromagnetic energy in an optical band between 400 nm and 12 nm.

3. The apparatus of claim 2, wherein electromagnetic energy is transmitted to remove the hair follicle from among the plurality of hair follicles.

4. The apparatus of claim 3, wherein the electromagnetic energy elevates the temperature of the hair follicle to be removed.

5. The apparatus of claim 3, further comprising an optical switch configured to switch the electromagnetic energy on or off.

6. The apparatus of claim 5, wherein each proximal end of each optical fiber of the plurality of multimode optical fibers is operatively connected to the optical switch.

7. The apparatus of claim 1, further comprising an electro-mechanical shutter synchronized with an on-off pulse of the light source.

8. The apparatus of claim 1, wherein the plurality of multimode optical fibers form a coherent fiber bundle.

9. The apparatus of claim 1, wherein the plurality of multimode optical fibers are configured in a ring surrounding the coherent imaging fiber.

10. The apparatus of claim 1, further comprising a cylindrical housing surrounded by the plurality of multimode optical fibers.

11. The apparatus of claim 10, wherein the cylindrical housing is configured to provide coolant to cool the coherent imaging fiber.

12. The apparatus of claim 11, wherein the cylindrical housing is constructed of stainless steel.

13. The apparatus of claim 1, wherein the applicator is remote from the light source.

14. The apparatus of claim 1, wherein the applicator is configured as a hand piece.

15. The apparatus of claim 1, wherein the viewing on the display is provided in real-time.

16. A hair removal method, the method comprising:
positioning an applicator configured to house proximal ends of each optical fiber of a plurality of multimode optical fibers and a proximal end of a coherent imaging fiber above a plurality of hair follicles;
viewing, via the coherent imaging fiber, an image of a hair follicle to be removed from among a plurality of hair follicles; and
transmitting, from a light source via the plurality of multimode optical fibers, energy to the hair follicle to be removed from among the plurality of hair follicles.

17. The method of claim 16, further comprising cooling the coherent imaging fiber by providing coolant via a housing adjacent to the coherent imaging fiber.

18. The method of claim 17, wherein the cylindrical housing is surrounded by the plurality of multimode optical fibers.

19. The method of claim 17, wherein the energy elevates temperature of the hair follicle to be removed.

20. The method of claim 17, wherein the applicator is configured as a hand piece, remote from the light source, and the viewing is provided in real-time.
